(19) United States
(12) Patent Application Publication
(10) Pub. No.: US 2004/0191290 A1
(43) Pub. Date: Sep. 30, 2004

(54) Solid pest control system

(75) Inventor: Donald W. Kelley, Athens, TX (US)

Correspondence Address: JACQUELINE S LARSON, P O BOX 2426, SANTA CLARA, CA, 95055-2426, US

(73) Assignee: Redline, Inc.

(21) Appl. No.: 10/805,926

(22) Filed: Mar. 22, 2004

Related U.S. Application Data
(60) Provisional application No. 60/458,682, filed on Mar. 28, 2003.

(51) Int. Cl. A61K 9/14; A01N 25/00

(52) U.S. Cl. 424/405; 424/486

(57) ABSTRACT

The present invention is directed to a pest control system comprising a low melting point polymer or copolymer, high levels of a solid fatty acid, and active compounds to produce a solid slow release generator of the active compounds.

This application claims the benefit of U.S. Provisional patent application Serial No. 60/458,682, filed Mar. 28, 2003, the entire disclosure of which is incorporated herein by reference.

### FIELD OF THE INVENTION

The present invention is directed to a pest control system comprising a low melting point polymer or copolymer, high levels of a solid fatty acid, and active compounds to produce a solid slow release generator of the active compounds.

### BACKGROUND OF THE INVENTION

It is well known that high molecular weight fatty acids such as stearic acid will bloom to the surface when compounded into a plastic matrix. Stearic acid is used in polymers as a process lubricant and an anti-block agent because of this property. However, when more than one to two percent of stearic acid is used with a conventional polymer, the compound becomes very difficult or impossible to process on conventional equipment such as an extruder or injection molding machine. This is due to the vast differences in melting points of stearic acid and the polymers plus the incompatibility between the two materials. The stearic acid lubricates to the extent that the compound simply turns in the barrel of the extruder or molding machine. If a low melt polymer conventional pellet is used, the same incompatibility is demonstrated when higher levels (above about 2%) of stearic acid is used.

### SUMMARY OF THE INVENTION

It has now been discovered by the applicant that by using small granules, small irregularly shaped particles or powder form of a low melting-point polymer, one is able to obtain a polymeric system containing high levels of a solid fatty acid, such as stearic acid. More particularly, the present invention is directed to a pest control system comprising a pest control formulation comprising a low melting polymer or copolymer (that is, a polymer or copolymer having a melt temperature of below 250° F., preferably below 200° F.), high levels of a solid fatty acid, and one or more active agents to produce a solid slow release generator of the active agents. By “high levels of a solid fatty acid” is meant from about 5 wt % to about 50 wt %, preferably from about 15 wt % to about 30 wt %, of the total formulation. The system is useful for making articles such as animal collars, ear tags, pest strips or blocks, and the like, for releasing the active agent from the article over an extended or prolonged period of time. By “extended or prolonged period of time” is meant for a period of activity longer than the period of activity exhibited by the raw active ingredient alone.

The present invention is further directed to a method for preparing a polymeric pest control system comprising high levels of a solid fatty acid, the method comprising combining an active agent with from about 5 wt % to about 50 wt % of the solid fatty acid, heating the combination to a liquid state, and then adding the combination to granules of, small irregularly shaped particles of, or powder of a low melting polymer or copolymer to make a dry blend. This dry blend may then be processed into a shape on a conventional extruder or molding machine at low temperatures. The resulting active agent generator is formed by extrusion or molding the mix into any desired shape such as a flea and tick collar for animals, a film covering for preventing bacteria or fungal growth on beds, etc.

### DETAILED DESCRIPTION OF THE INVENTION

As used herein, “a” and “an” mean one or more, unless otherwise indicated.

The “fatty acid” or “solid fatty acid” useful in the present invention is any fatty acid of from about 16 to about 36 carbon atoms and being a solid at room temperature. Such fatty acids include, but are not limited to, stearic, palmitic, margaric, nonadecanoic, arachidic, heneicosanoic, dehenic, tricosanoic, tetracosanoic, pentacosanoic, and cerotic fatty acids. The solid fatty acid is present in the formulation of the invention in an amount of from about 5 wt % to about 50 wt %, preferably from about 15 wt % to about 30 wt %.

The “low melting polymer or copolymer” is selected from those polymers or copolymers having a melt temperature of below 250° F., preferably below 200° F. Examples of polymers and copolymers useful in the present invention include, but are not limited to, polyethylene, polyvinyl acetate, polyethylene, ethylene acid copolymers, ethylene acrylates, polyurethanes, styrene-butadiene, polyvinyl acetate, polyvinyl butyral, and mixtures and copolymers thereof. In order to obtain compatibility with the solid fatty acid and to improve processing of the formulation in conventional equipment, the polymer or copolymer is used, partially or wholly, in the form of granules, small irregularly shaped particles or powder. Conventional pellets of polymers are very difficult to work with in this invention and can only constitute a small percentage (no greater than about 15 wt % and preferably below about 10 wt %) of the total amount of polymer or copolymer, if they are used at all. The low melting polymer or copolymer is present in the formulation of the invention in an amount of from about 40 wt % to about 80 wt %, preferably from about 50 wt % to about 70 wt %, of which at least about 40 wt % is in the form of granules, small irregularly shaped particles or powder.

The pest control active agent may be an insecticide, bactericide, fungicide, acaricide, attractant, repellent, or any other biologically active ingredient that is compatible with the other components of the pest control system. In one presently preferred embodiment, the active agent is chosen from any active agent known to be useful in the control of insect or acarid pests. Exemplary pesticides and repellents which are effective against horn flies, face flies, stable flies, house flies, mosquitoes, lice, ticks, and mites are bioresmethrin, permethrin, tetramethrin, cypermethrin, decamethrin, pyrethrins, resmethrin, cyhalothrin, allethrin, dichlorvos, carbaryl, naled, citrus oils, citronella oil, pine oil, stirofos, fenvalerate, stabilene, benzyl benzoate, methyl nonyl ketone, N-butylacetanilide, di-n-propyl isocinchomeronate, 2-octylthioethanol, dimethyl carbate, dimethyl phthalate, N,N-diethyl-m-toluamide, and 2,3:4,5-bis (2-butylene)-tetrahydro-2-furfural. Many of these active ingredients are effective both as a pesticide and as a repellent, and the activity of many is enhanced by the inclusion of a synergist. Especially preferred synergists include piperonyl butoxide and N-octyl bicycloheptene dicarboximide. The active agent may be a liquid or a solid at room temperature.

To prepare pest control systems according to the invention, the pest control active agent and the solid fatty acid are mixed together at a predetermined ratio, with the proviso that the fatty acid is present at a high level. Generally, the amount of fatty acid in the formulation should be at least about 5 wt %, preferably at least about 15 wt %. The active agent/fatty acid mixture is then heated to a liquid state and added to granules of, small irregularly shaped particles of, or powder form of a low melting polymer or copolymer to make a dry blend. This dry blend formulation may then be processed into a shaped article, such as a pet collar or an ear tag or the like, on a conventional extruder or molding machine at low temperatures (that is, at temperatures that will melt the low melting polymer or copolymer, which is generally below about 250° F.) by methods known in the art.

If processing of the shaped article takes place at higher temperatures, the article should be cooled to room temperature as quickly as possible. In some cases when the article is not quickly cooled, there is excessive bloom on the surface, which can flake off. When this happens, the article can be annealed at 140° F. as a post operation to prevent the excessive bloom.

Additional components may optionally be included in the pest control system of the invention. Such optional ingredients can include, but are not limited to, plasticizers, synergists, fragrances, coloring agents, preservatives, antioxidants, light stabilizers, and the like.

After being processed into the desired shape, the active agent will, together with the solid fatty acid, bloom to the surface of the article, making the active agent available to an environment, such as an animal for example, for pest control purposes, such as, for example, the control of insects and/or acarids on the animal. The fatty acid/active agent combination blooms to the surface and stops until a part of the surface material is removed. When the surface material is removed, it is replaced by more of the combination of the fatty acid/active agent from the inter-matrix of the plastic.

The following examples illustrate the practice of the present invention. Parts are given as percentages and temperature in degrees Fahrenheit unless otherwise noted. “RT” is room temperature.

### EXAMPLES

### Example 1

The formulation in Table 1 is prepared, and is then formed into an insecticidal dog collar, as follows:

TABLE 1

| Ingredients: | Percentages: |
|---|---|
| d-cyphenothrin (Gokilaht) Tech. | 15.6 |
| Safflower Oil | 5.0 |
| Stearic Acid | 20.0 |
| Polymer MU 760-00 | 59.3 |
| Blaze Orange T-15 colorant | 0.1 |

Sources:

Gokilaht (d-cyphenothrin; synthetic pyrethroid) Technical—MGK Company.

Microthene® Polymer MU 760-00 (ethylene-vinyl acetate copolymer, ground powder, melt index: 32 (EMI), particle size: 35 mesh)—Equistar Chemicals, LP.

Colorant—Day Glow Color Corp

Mixing Procedure:

1. The Gokilaht, safflower oil and stearic acid are weighed together. Heat is applied and the mixture brought to a liquid state at 165° F.

2. The polymer is weighed and placed into a mixing vessel.

3. The liquid active agent/stearic acid mixture is slowly added to the polymer while mixing.

4. The Blaze Orange is then added and the resulting blend is allowed to cool to room temperature.

The blend is then extruded or molded into the shape desired, which, in this Example was a dog collar.

These collars were subjected to efficacy evaluation against fleas and ticks. The tests consisted of a treated group of three dogs (one collar per dog) and a control group of three untreated dogs. The dogs were chosen from random breed adult dogs of mixed sexes and with reasonably uniform haircoat types, and the dogs were individually housed, fed and maintained. The dogs were treated once on day 0 by buckling the test collar around the dogs' necks, leaving at least space for 2 fingers. The dogs were infested with fleas (Ctenocephales felis) and ticks (Rhipicephalus sanguineus) on the day before treatment and then re-infested weekly thereafter, each re-infestation to be made approximately 24 hours before the first of the next series of flea and tick counts. Flea and tick timed finger counts were performed at 24 and 48 hours after treatment and at 24 hours after each re-infestation. Comb counts, by removing and discarding all fleas and ticks, were performed at 72 hours after treatment and after each re-infestation.

The results are presented in Tables I-A and I-B below:

TABLE I-A
Three-Dog Group Mean Efficacy Against Fleas

| Day | % Efficacy |
|---|---|
| 1 | 53 |
| 2 | 71 |
| 3 | 86 |
| 7 | 79 |
| 9 | 85 |
| 14 | 76 |
| 16 | 87 |
| 21 | 91 |
| 23 | 96 |
| 28 | 79 |
| 30 | 94 |
| 35 | 84 |
| 37 | 91 |
| 43 | 84 |
| 45 | 91 |

TABLE I-B
Three-Dog Group Mean Efficacy Against Ticks

| Day | % Efficacy |
|---|---|
| 1 | 70 |
| 2 | 74 |
| 3 | 76 |
| 7 | 80 |
| 9 | 84 |
| 14 | 90 |
| 16 | 91 |
| 21 | 95 |
| 23 | 96 |
| 28 | 97 |
| 30 | 95 |
| 35 | 91 |
| 37 | 95 |
| 43 | 98 |
| 45 | 99 |

### Example 2

The formulation in Table 2 is prepared, following the procedures of Example 1:

TABLE 2

| Ingredients: | Percentages: |
|---|---|
| Propoxur (Sendran) Tech. | 15.60 |
| Nylar, 97% acitve | 0.7 |
| Safflower Oil | 10.0 |
| Stearic Acid | 20.0 |
| Polymer MU 760-00 | 58.6 |
| Rocket Red colorant | 0.1 |

Sources:

Sendran (2-isopropoxyphenyl-N-methylcarbamate) Technical—Bayer, Inc.

Nylar® comprises approximately 50% by weight pyriproxyfen and approximately 50% by weight corn oil and is available from MGK Company.

### Example 3

The formulation in Table 3 is prepared, following the procedures of Example 1:

TABLE 3

| Ingredients: | Percentages: |
|---|---|
| Permethrin Tech. | 15.6 |
| Nylar, 97% active | 0.7 |
| Safflower Oil | 5.0 |
| Stearic Acid | 20.0 |
| Polymer MU 760-00 | 58.6 |
| Blue Pigment R6BL9019 | 0.1 |

Sources:

Permethrin Technical—MGK Company.

The material of the above formulation was subjected to a weigh loss test to ascertain if the non-polymer materials would release from the polymer matrix. The test consisted of extruding the material into dog collars, weighing the collars, wiping the surface of the collars with a clean paper towel and reweighing. The weight difference from each wiping demonstrates the weight loss as it might happen in actual use. A commercial cat pest control collar was similarly tested, as a comparison. While the weight loss of the collar of this invention was lower overall than that of the commercial collar, it followed the same profile of continuous loss over the course of the 25-day study. Also, while it was impossible in this study to ascertain the percent of active agents being released by the wipe test, visual observations show that the released material was a mixture of the oil phase materials and stearic acid.

### Example 4

The formulation in Table 4 is prepared, and is then formed into an insecticidal dog collar, as follows:

TABLE 4

| Ingredients: | Percentages: |
|---|---|
| d-cyphenothrin (Gokilaht) Tech. | 14.2 |
| Phosflex 390 | 5.1 |
| Stearic Acid | 6.4 |
| Nylar, 98.8% active | 0.6 |
| Polymer MU 760-00 | 53.7 |
| Elvax 150 | 10.0 |

Sources:

Phosflex 390 (isodiphenyl phosphate)—Akzo Nobel.

Elvax (ethylene-vinyl acetate copolymer; conventional pellets)—DuPont.

Mixing Procedure:

1. The Gokilaht, phosflex, stearic acid and Nylar are weighed and added to a heatable mixing vessel. The materials are heated to 165° F. and mixed until a honogenous solution is achieved.

2. The MU 760-00 and the Elvax 150 polymers are weighed, placed into a mixing vessel and blended to uniformity.

3. While the polymers are mixing, the heated liquid active agent/stearic acid mixture is sprayed onto the polymers. Mixing continued until the mass reached room temperature.

4. The liquids should be applied to the polymer powder while the polymers are being mixed, so as not to cause large lumps to form. This should be done far enough in advance of extrusion (generally about 24 hours is sufficient) so that the liquids can solidify and a free-flowing powder is achieved.

The resulting formulation was extruded into dog collars. The Extrusion Profile is:

Extruder: Prodex 2½ inch; PE Screw 24/1 single stage; Screw rpm = 30

Zone temperatures:

| | | | | | | |
|---|---|---|---|---|---|---|
| #1 | #2 | #3 | #4 | #5 | Gate | Die |
| off | off | off | 205 | 205 | 195 | 195 |

Screen Pack:
1-40
2-80

Collar dimensions and weight: 1 in. × 0.513 in. × 0.109 in. −0.748 grams;
oval shape

What is claimed is:

1. A polymeric formulation comprising from about 40 wt % to about 80 wt % of a low melting polymer or copolymer, from about 5 wt % to about 50 wt % of a solid fatty acid, and one or more active agents.

2. A polymeric formulation according to claim 1 which comprises from about 40 wt % to about 80 wt % of low melting polymer or copolymer originally in the form of granules, small irregularly shaped particles or powder, and from zero to about 15 wt % of low melting polymer or copolymer originally in the form of conventional pellets, with the proviso that the total amount of low melting polymer or copolymer in the formulation is no greater than about 80 wt %.

3. A polymeric formulation according to claim 1 wherein the low melting polymer or copolymer is ethylene-vinyl acetate copolymer.

4. A polymeric formulation according to claim 2 wherein the low melting polymer or copolymer is ethylene-vinyl acetate copolymer.

5. A polymeric formulation according to claim 1 wherein the solid fatty acid is stearic acid.

6. A polymeric formulation according to claim 3 wherein the solid fatty acid is stearic acid.

7. A polymeric formulation according to claim 1 wherein the active agent is selected from those that are useful in the control of insect or acarid pests.

8. A polymeric formulation according to claim 1 wherein the active agent is selected from the group consisting of insecticides, bactericides, fungicides, acaricides, attractants, repellents, and biologically active ingredients.

9. A polymeric formulation according to claim 1 which further comprises an active agent synergist.

10. A method for synthesizing a polymeric pest control system, the method comprising:
mixing together at least one active agent and a solid fatty acid, the solid fatty acid being in an amount of from about 5 wt % to about 50 wt % of the final formulation;
heating the active agent/fatty acid mixture to a liquid state;
adding the active agent/fatty acid mixture to a low melting polymer or copolymer to make a dry blend, the polymer or copolymer being in an amount of from about 40 wt % to about 80 wt % of the final formulation; and
processing or forming the dry blend into the shape of the pest control system.

11. A method according to claim 10 wherein the polymer or copolymer is in an amount of from about 40 wt % to about 80 wt % in the form of granules, small irregularly shaped particles or powder, and from zero to about 15 wt % of low melting polymer or copolymer in the form of conventional pellets, with the proviso that the total amount of low melting polymer or copolymer in the formulation is no greater than about 80 wt %.

12. A method according to claim 10 wherein the low melting polymer or copolymer is ethylene-vinyl acetate copolymer.

13. A method according to claim 10 wherein the solid fatty acid is stearic acid.

14. A method according to claim 12 wherein the solid fatty acid is stearic acid.

15. A method according to claim 10 which further comprises adding an active agent synergist to the active agent/fatty acid mixture.

16. A pest control system comprising a polymeric formulation according to claim 1.

17. A pest control system according to claim 16 which is selected from the group consisting of animal collars, ear tags, pest strips, pest blocks, and film coverings.

18. A pest control system which is an animal collar comprising ethylene-vinyl acetate copolymer, from about 5 wt % to about 50 wt % of stearic acid, and from about 40 wt % to about 80 wt % of low-melting polymer or copolymer.

19. A method for releasing an active agent into an environment over an extended period of time, the method comprising placing a pest control system into the environment, wherein the pest control system comprises a polymeric formulation according to claim 1.

20. A method according to claim 19 wherein the pest control system is an animal collar comprising ethylene-vinyl acetate copolymer and stearic acid.
